(19) United States
(12) Patent Application Publication
(10) Pub. No.: US 2005/0267368 A1
(43) Pub. Date: Dec. 1, 2005

(54) Ultrasound strain imaging in tissue therapies

(75) Inventors: Emad M. Boctor, Baltimore, MD (US); Gabor Fichtinger, Bethesda, MD (US)

Correspondence Address: MCKENNA LONG & ALDRIDGE LLP, 1900 K STREET, NW, WASHINGTON, DC, 20006, US

(73) Assignee: THE JOHNS HOPKINS UNIVERSITY, Baltimore, MD (US)

(21) Appl. No.: 11/124,116

(22) Filed: May 9, 2005

Related U.S. Application Data
(63) Continuation-in-part of application No. 10/895,397, filed on Jul. 21, 2004.
(60) Provisional application No. 60/569,003, filed on May 7, 2004.
(60) Provisional application No. 60/577,789, filed on Jun. 8, 2004.
(60) Provisional application No. 60/488,941, filed on Jul. 21, 2003.

(51) Int. Cl. A61B 8/00

(52) U.S. Cl. 600/443; 600/438

(57) ABSTRACT

Disclosed is a system and method for providing ultrasound strain imaging of a soft tissue, such as a prostate, so that the soft tissue may be more precisely targeted during radiation treatment. The system includes a mechanical arm with a pressure interface for applying incremental pressure to the patient's abdomen, and a data system for correlating the ultrasound signals acquired before and after each application of incremental pressure. By correlating the pre and post-pressure ultrasound signals, acoustic interfaces corresponding to the prostate, which define the contours of the prostate, may be displayed. Further, data corresponding to the contours of the prostate may be provided to a linear accelerator to enable the linear accelerator to more precisely target the prostate.

This application claims the benefit of U.S. Provisional Patent Application No. 60/569,003, filed on May 7, 2004; U.S. Provisional Patent Application No. 60/577,789, filed on Jun. 8, 2004; and U.S. Non-provisional Application Ser. No. 10/895,397 titled ROBOTIC 5D ULTRASOUND SYSTEM, which claims priority to U.S. Provisional Patent Application No. 60/488,941, filed Jul. 21, 2003, all of which are hereby incorporated by reference for all purposes as if fully set forth herein.

The research and development effort associated with the subject matter of this patent application was supported by the National Science Foundation under grant no. EEC9731478.

### BACKGROUND OF THE INVENTION

1. Field of the Invention

The present invention relates primarily to the field of radiation oncology. More particularly, the present invention involves a system and method to provide more precise targeting of radiation by improving ultrasound imaging of the target tissue.

2. Discussion of the Related Art

Linear accelerators are commonly used in the treatment of cancer, particularly breast and prostate cancer. Breast cancer and prostate cancer patients typically undergo 25 and 40 radiation treatments, respectively, as part of a treatment regimen. In providing radiation treatment, linear accelerators typically deliver electromagnetic radiation dosages of energies on the order to of 20 million electron volts into a target area. Given such high energieslevels, there is a need to precisely deliver that energy into the target the (e.g., the tumor) in order maximize the dosage delivered to the tumor while minimizing radiation exposure to healthy tissue.

In order to precisely target the tumor, it is necessary to know its location while the patient is lying on the linear accelerator table. Various imaging techniques have been developed to meet this need. One method involves the use of Electronic Portal Image Detectors (EPIDs), which are integrated into the linear accelerator. EPIDs and provide X-Ray images of the target area while the patient is lying on the linear accelerator table. However, oOne problem with this approach is that EPIDs is that they do not provide imagery of soft tissue. Instead, EPIDs typically provide imagery of bone in the vicinity of the target tissue, which the linear accelerator technician may use as a “landmark” references for estimating the location of the tumor.

As used herein, the term “tumor” generally refers to any target feature within a patient. As used herein, The term “imagery” generally refers to one or more images acquired by an imagining system, such as a CT, MRI, and or ultrasound system. However, imagery may also refer to data values in “raw” electronic form, such as binary data, or may refer to one or more images displayed on a computer screen or printed in a hardcopy form.

To facilitate the description of the present invention, the present invention is described below with reference to The discussion below refers to the treatment of prostate cancer. However, it will be apparent to one of ordinary skill where the parallels lie between the treatment of prostate cancer and the treatment of other cancers, such as that the present invention is applicable to other treatments, such as breast cancer.

Another imaging method for assisting in the targeting of tumors the tumor involves the use of transcutaneous ultrasound, in which a linear accelerator technician applies a transcutaneous ultrasound probe onto the patient's abdomen between radiation dosages. The problem with this approach is that intervening acoustic interfaces, such as the bladder, air pockets in the bladder, bone, etc., make such images unreliable for locating the tumor. Related art systems that use this approach include ultrasound systems manufactured by NOMOS Corp., Varian Corp., Computerized Medical Systems, Inc., and Resonnant Corp.

Related Yet other related art solutions have emerged that provide for localization of the prostate. One such solution involves implanting electromagnetic transponders into the prostate, which are then located by use of using a radar-like scanning mechanism, such as the Calypso® product line by Calypso® Medical Technologies, Inc. of Seattle Wash. Still other approaches include implanting gold markers, which are visible in X-Ray imagery made by using EPIDs. A disadvantage of such solutions is that the implanted devices are mere surrogates for the prostate, and they which do not provide information regarding that specifically defines the contours of the prostate. In addition, these devices, and which must be invasively implanted. Further, swelling of the prostate may occur during treatment, which could cause displace the surrogate implants to move relative to the location of the tumor. Either way, targeting by use of surrogates involves the use of foreign objects placed in the vicinity of the tumor whereby the linear accelerator technician does not actually see the prostate. The technician, but must therefore estimate its the contours of the prostate and the location of the tumor relative to the surrogates.

### SUMMARY OF THE INVENTION

Accordingly, the present invention is directed to ultrasound strain imaging in tissue therapies ultrasound strain imaging in prostate therapies that substantially obviatess one or more of the aforementioned problems due to limitations and disadvantages of the related art. In general, the present invention achieves this by providing an image imagery corresponding to that contains the contours of the targeted anatomical structure (e.g., the prostate). The present invention provides this imagery by providing increasing pressure in the vicinity of the targeted anatomical structure while acquiring ultrasound images, identifying boundaries of differential strain as revealed in the images, and reconstructing the contours of the targeted anatomical structure from the images.

An advantage of the present invention is to assist in maximizing that it maximizes radiation dosages to tumors while reducing the exposure to of surrounding healthy tissue to the radiation.

Another advantage of the present invention is to that it provides more accurate imagery of a prostate the targeted anatomical structure (e.g., the prostate) during radiation therapy, which it does by taking advantage of the differences in strain properties between the targeted anatomical structure and prostate and its surrounding tissue.

Additional features and advantages of the invention will be set forth in the description which follows, and in part will be apparent from the description, or may be learned by practice of the invention. The objectives and other advantages of the invention will be realized and attained by the structure particularly pointed out in the written description and claims hereof as well as the appended drawings.

To achieve these The aforementioned and other advantages and in accordance with the purpose of the present invention are achieved with, a system that provides for providing ultrasound strain imaging. The system comprises an ultrasound probe; a mechanical arm connected to the ultrasound probe; an ultrasound processor; and a data system having a computer readable medium encoded with a program for controlling the mechanical arm to apply pressure on a patient's abdomen anatomy, and for correlating ultrasound signals acquired by the ultrasound probe.

In another aspect of the present invention, the aforementioned and other advantages are achieved by a method for identifying a contour corresponding to a target anatomical structure in ultrasound imagery. The method involves positioning an ultrasound probe to be substantially in contact with a patient's anatomy, the ultrasound probe being connected to a mechanical arm; acquiring a first ultrasound signal from the ultrasound probe; commanding a mechanical arm to apply pressure to the anatomy; acquiring a second ultrasound signal from the ultrasound probe; correlating the first ultrasound signal with the second ultrasound signal; identifying a contour based on the result of the correlating; and displaying the contour.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

FIG. 1 illustrates an exemplary ultrasound strain imaging apparatus system according to the present invention;

FIG. 2 illustrates an exemplary ultrasound strain imaging apparatus system, which includes being used in conjunction with a linear accelerator for for prostate cancer treatment;

FIG. 3 illustrates a bladder and a prostate subject to strain, along with multiple fields of view projected by an ultrasound probe;

FIG. 4A illustrates exemplary echo signals for given field of view with and without strain exerted on the prostate;

FIG. 4B illustrates exemplary echo signals for a different field of view with and without strain exerted on the prostate;

FIG. 5 illustrates another exemplary ultrasound strain imaging apparatus system using an second ultrasound transmitter probe and integrated with a rectal balloon; and

FIG. 6 illustrates an exemplary process for performing ultrasound strain imaging according to the present invention.

### DETAILED DESCRIPTION OF THE ILLUSTRATED EMBODIMENTS

As used herein, ultrasound strain imaging refers to acquiring ultrasound imagery of an anatomical region, which includes a targeted anatomical structure and its surrounding tissue, region while precisely incrementally applying incremental amounts of substantially precise pressure against an area of patient's body in the vicinity of the anatomical region abdomen. The applied pressure exerts strain on the tissue within the abdomen anatomical region, including the targeted anatomical structure and prostate and its surrounding tissue. The surrounding tissue and the targeted anatomical structure prostate respond differently to the exerted strain such that the tissue and the targeted anatomical structure prostate will compress at different rates. By correlating the ultrasound data acquired with and without strain, the contours of the targeted anatomical structure prostate may be identified based on its different response to the strain.

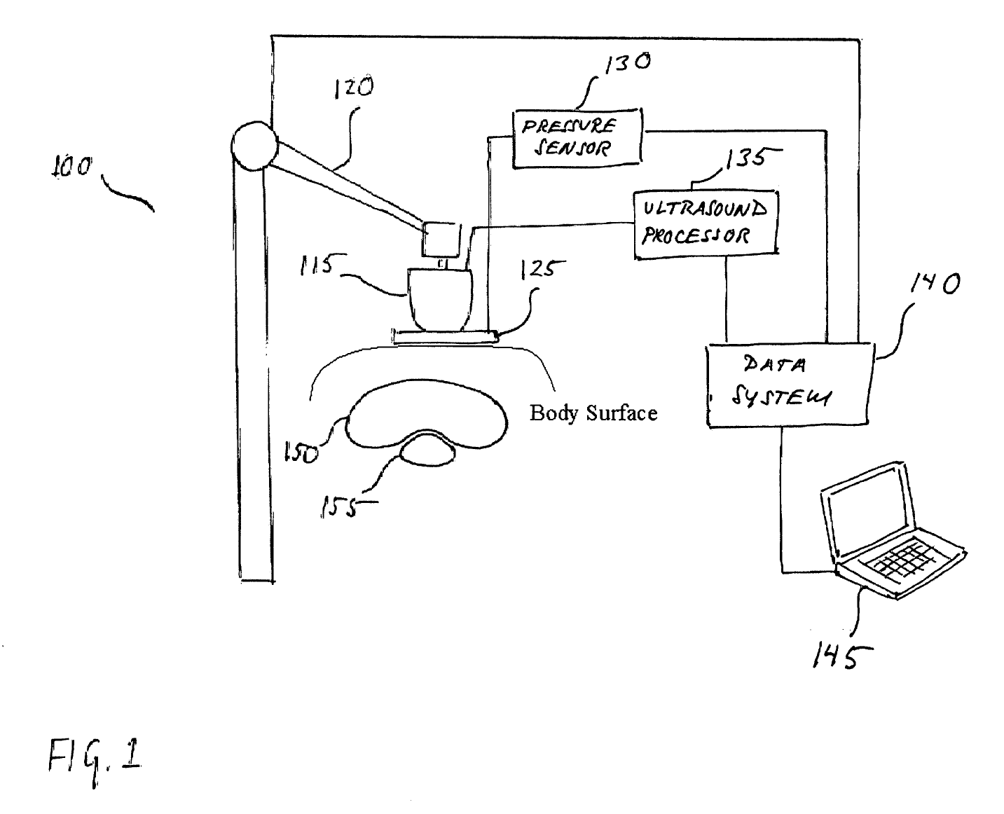

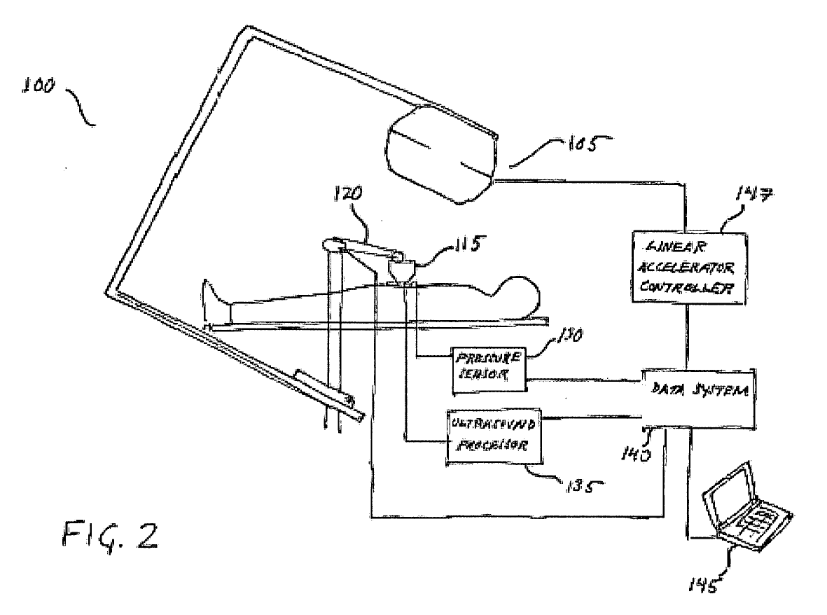

For purposes of illustration and not limitation, the present invention as illustrated in FIGS. 1 and 2 is described in terms of a system for providing images of the prostate. However, it will be readily evident to those skilled in the art that the imaging system described herein below and illustrated in FIGS. 1 and 2 could be employed to provide images of anatomical structures other than the prostate.

FIG. 1 illustrates an exemplary strain imaging system 100 according to the present invention. The system 100 includes a transcutaneous ultrasound probe 115; a pressure interface 125 connected to the transcutaneous ultrasound probe 115; a mechanical arm 120 connected to the transcutaneous ultrasound probe 115; a pressure sensor 130 connected to the pressure interface 125; an ultrasound processor 135 for providing and receiving signals and data to and from the transcutaneous ultrasound probe 115; a data system 140; and a user interface 145.

FIG. 2 illustrates an exemplary strain imaging system 100, as described above, further including a linear accelerator 105, and a linear accelerator controller 147 connected to the data system 140.

The transcutaneous ultrasound probe 115 may have a plurality of elements (referred to herein as “N” elements) for providing imagery of the bladder 150 and the prostate 155 within the patent. The ultrasound probe may be connected to the mechanical arm 120, or may be held and applied to the patient manually. For the purposes of illustration, this embodiment of the present invention may employ a Siemens C7F2 3D “wobbler” probe for the transcutaneous ultrasound probe 115, which is manufactured by Siemens Medical Solutions, USA, Inc., Ultrasound Division, Issaqua, Wash. However, it will be readily apparent to one skilled in the art that other commercially available ultrasound probes may be used. The transcutaneous ultrasound probe 115 may operate in pulse/echo mode, whereby the probe transmits acoustic energy and detects the reflected acoustic energy.

The pressure interface 125 may have a substantially flat lower surface for applying substantially planar pressure against the patient's abdomen. The pressure interface 125, which is connected to the transcutaneous ultrasound probe 115 may have an opening through which the transcutaneous ultrasound probe 115 may be inserted for contacting the patient. Alternatively, the pressure interface may be integrated into the transcutaneous ultrasound probe 115 and be acoustically coupled to the probe. Depending on the shape of the transcutaneous ultrasound probe 115, if the probe is capable of providing substantially planar pressure against the abdomen, the pressure interface 125 may not be necessary.

The data system 140 may include one or more computers, which may be connected together either locally or over a network. The data system 140 includes a memory encoded with software (hereinafter “the software”) for implementing processes according to the present invention. The software may be stored and run on the data system 140, or may be stored and run in a distributed manner between the data system 140 the ultrasound processor 135, and the user interface 145.

The pressure sensor 130 measures the pressure exerted by the pressure interface 125 against the patient's abdomen. The pressure sensor 130 may measure the pressure as exerted by the pressure interface 125, or may that exerted by the transcutaneous ultrasound probe 115, if the pressure interface 125 is not used. The pressure sensor 130 is connected to the data system 140 so that pressure measurements made by the pressure sensor 130 may be acquired and stored by the software.

The ultrasound processor 135 is for sending control signals to, and receiving data from, the transcutaneous ultrasound probe 115. For the purposes of illustration, this embodiment of the present invention may employ a SONOLINE™ Antares ultrasound scanner system for the ultrasound processor 135, which is manufactured by Siemens Medical Solutions, USA, Inc., Ultrasound Division, Issaqua, Wash. for the ultrasound processor 135, and may employ a Siemens C7F2 3D “wobbler” probe for the transcutaneous ultrasound probe 115, which may be held in a rigid attachment mounted to the mechanical arm 120. However, it will be readily apparent to one skilled in the art that other commercially available ultrasound scanners and probes processors may be used. The ultrasound processor 135 is connected to the data system 140 for receiving control signals and providing ultrasound image data.

The mechanical arm 120 may provide at least one degree of freedom of motion to the transcutaneous ultrasound probe 115 and the pressure interface 125. The mechanical arm 120 is connected to the data system 140 for receiving control signals and sending data regarding its position, orientation, rate of motion, and acceleration for each degree of freedom. The mechanical arm 120 may be controlled by commands issued by an operator via the user interface 145. The mechanical arm 120 may also be controlled robotically by the software using motion control algorithms that are known to the art.

The pressure interface 125, under force provided by the mechanical arm 120, provides substantially homogeneous and repeatable pressure of the patient's abdomen, which in turn exerts strain on the bladder 150 and prostate 155. Depending on the shape of the transcutaneous ultrasound probe 115, and the uniformity of the pressure it applies, a separate pressure interface 125 may not be necessary.

The data system 140 may include one or more computers, which may be connected together either locally or over a network. The data system 140 includes a memory encoded with software (hereinafter “the software”) for implementing processes according to the present invention. The software may be stored and run on the data system 140, or may be stored and run in a distributed manner between the data system 140 the ultrasound processor 135, and the user interface 145.

The user interface 145 may include one or more computers that communicate with the data system 140. The user interface may also include computers that are connected remotely over a network to allow for remote operation and monitoring of operation.

The linear accelerator 105 may be one of a number of models that are used in cancer treatment, which have the capability of fine-tuning the focusing of radiation based on commands issued by the linear accelerator controller 147. The linear accelerator controller 147 may have data inputs for accepting commands from the data system 140, or may have a separate user interface by which commands may be entered manually.

FIG. 2 illustrates system 100 being used in conjunction with a linear accelerator 105 in performing prostate cancer treatment. The transcutaneous ultrasound probe 115 may be placed against the patient's abdomen so that it will not block the transmission of the linear accelerator 105, and may thus be used intra-operatively.

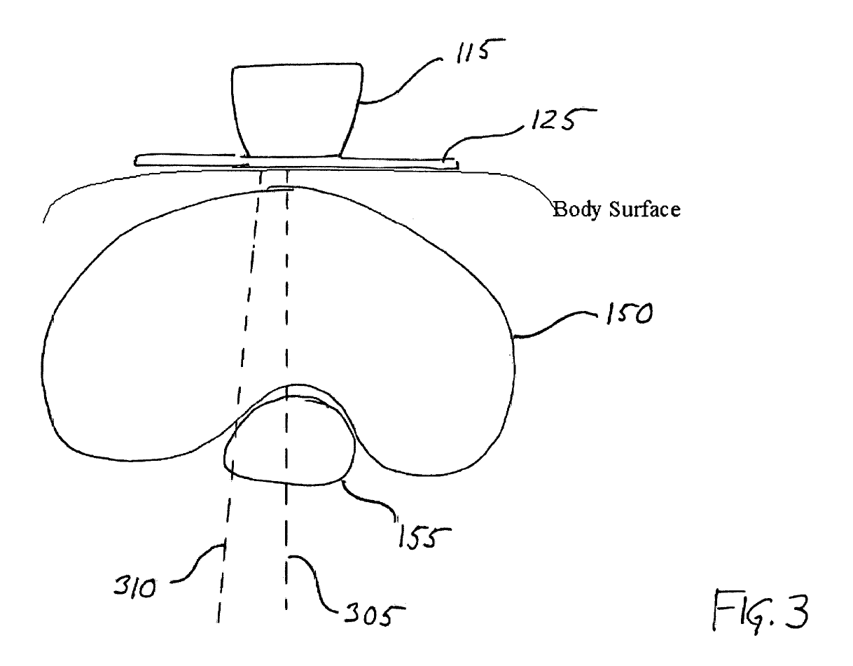

FIG. 3 illustrates a transcutaneous ultrasound probe 115 connected to a pressure interface 125. Together, the transcutaneous ultrasound probe 115 and the pressure interface 125, which are exerting strain on a patient's bladder 150 and a prostate 155. Also illustrated are two fields of view, represented by dashed lines 305 and 310. The two different fields of view may be achieved through the use of two different elements within the transcutaneous ultrasound probe 115, each of which correspond to different transducer elements in the transcutaneous ultrasound probe 115. Once the a strain from corresponding to a few millimeters to a few centimeters of displacement, depending on the patient, of approximately 1 cm is exerted on the bladder 150, the bladder 150 may conforms around the prostate 155, as illustrated in FIG. 3. The amount of strain required to make the bladder 150 conform around the prostate 155 may vary depending on the patient, the pressure within the bladder 150, and the size of the prostate 155. In conforming around the prostate 155, the bladder 150 substantially stabilizes the prostate 155 and may prevent it from moving. If the bladder 150 is full, the fluid within the bladder 150 provides for acoustic coupling between the transcutaneous ultrasound probe 115 and the prostate 155. Further, the fullness of the bladder 150 may help give it a degree of rigidity, which will help stabilize the prostate 155 when the pressure interface 125 exerts strain on the bladder 150.

Once the prostate 155 has been stabilized, further pressure exerted by the pressure interface 125 will continue to exert strain on the prostate 155. Once the prostate 155 experiences strain, up to approximately 1 mm, the contours of the prostate may become more visible in the imagery acquired by the transcutaneous ultrasound probe 115 and processed according to the present invention.

The pressure sensor 130, which is optional, may measure the pressure being exerted on the abdomen by the pressure interface 125. The pressure measurements may assist the operator in controlling the mechanical arm 120. Further, the pressure measurements may be acquired and stored by the software to provide additional information, along with the translation of the pressure interface 125, pertaining to the strain exerted on the bladder 150 and the prostate 155.

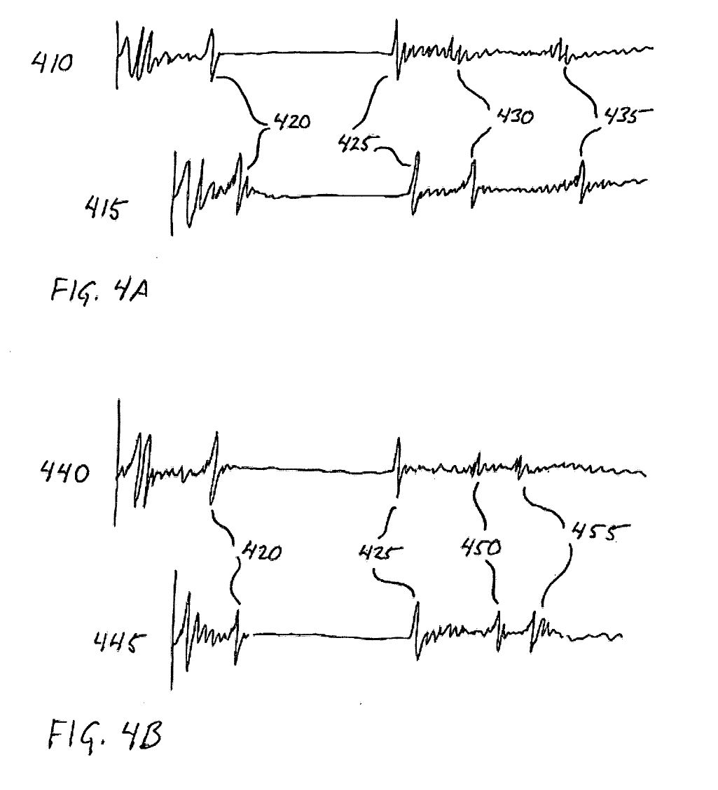

FIG. 4A illustrates two exemplary signals 410 and 420 from a single transcutaneous ultrasound probe 115 element having field of view 305; and FIG. 4B illustrates two exemplary signals 440 and 450 from a single transcutaneous ultrasound probe 115 element having field of view 310.

Referring to FIG. 4A and FIG. 3, signal 410 depicts the acoustic energy received by an element of the transcutaneous ultrasound probe 115 having field of view 305 when no pressure is applied by pressure interface 125 and the transcutaneous ultrasound probe 115. Signal 41520 depicts the acoustic energy received by the same element when pressure is being applied by pressure interface 125 and transcutaneous ultrasound probe 115, and the prostate 155 is under strain. Signals 410 and 415 have a signal response feature 420, which is in response to the echo of represents that portion of the acoustic energy emitted by transcutaneous ultrasound probe 115. Signal response feature 420 corresponds to the, which reflects back from acoustic interface 320 at the beginning that corresponds to the near surface of the bladder. The portion of signal 410 and signal 415 following signal feature 420 The signal following acoustic interface 320 is generally faint since it reflects the acoustic energy is propagating through water fluid in the bladder 150, which is substantially free of acoustic scatterers. Signal response feature 425 represents the energy reflected back from corresponds to the acoustic interface 325, which corresponds to the flat surface of at the far end of the bladder 150.

Signal response feature 430 corresponds to the represents the energy that reflects back from acoustic interface 330, which corresponds to the near surface at the beginning of the prostate 155. Similarly, and signal response feature 435 represents the energy reflected back from corresponds to the acoustic interface 335, which corresponds to the far surface of at the end of the prostate 155. Signal response features 430 and 435 may have an a sufficiently large amplitude that makes then them distinguishable from the background signal, as illustrated in FIG. 4A. However, this is not always the case. In this instance, these features may not have an amplitude that makes them distinguishable from the background, in which case speckle correlation (to be described later) may be required to identify these features in the form of temporal shifts in the ultrasound imagery that may be retrieved via correlation techniques that are known to in the art.

Referring to FIG. 4B and FIG. 3, signals 440 and 445 are respectively similar to signals 410 and 415, but correspond to acoustic energy received by an element of transcutaneous ultrasound probe 115 having field of view 310. Signal 440 represents the acoustic energy received when no pressure is being exerted by the transcutaneous ultrasound probe 115 and the pressure interface 125. Signal 445 represents the acoustic energy received when pressure is being applied by the transcutaneous ultrasound probe 115 and the pressure interface, and whereby the prostate 155 is under strain. Given the orientation of field of view 310, signal response features 450 and 455 respectively correspond to acoustic interfaces 330 and 335 (i.e., the near and far surfaces of the prostate 155). As illustrated in FIG. 4B, signal response features 450 and 455 are closer to each other, as compared to response features 430 and 435 of FIG. 4A. This is expected as the near and far surfaces of the prostate 155 are closer along field of view 310 than they are along field of view 305 corresponding to the manner in which field of view 335 “slices” prostate 155.

FIGS. 4A and 4B refer to signals detected by two elements of transcutaneous ultrasound probe 115. A transcutaneous ultrasound probe 115 may have N elements. Given multiple N elements, with each having a field of view that provides a different “slice” of prostate 155, there will be N signals similar to the signals illustrated in FIG. 4A. Each of the N signals may provide information regarding the near and far surfaces of the prostate 155, depending on the orientation of a particular element's field of view. The software may determine the near and far surfaces of the prostate 155 for each field of view, and then assemble the corresponding near and far surface information signal response features 450 and 455 to reconstruct the contours of the prostate 155. The value of N depends on the transcutaneous ultrasound probe 115 used. As transcutaneous ultrasound probe technology develops to include more elements, this may enable reconstructing the contours of the prostate 155 with greater spatial resolution, since there will be more fields of view intersecting the prostate 155.

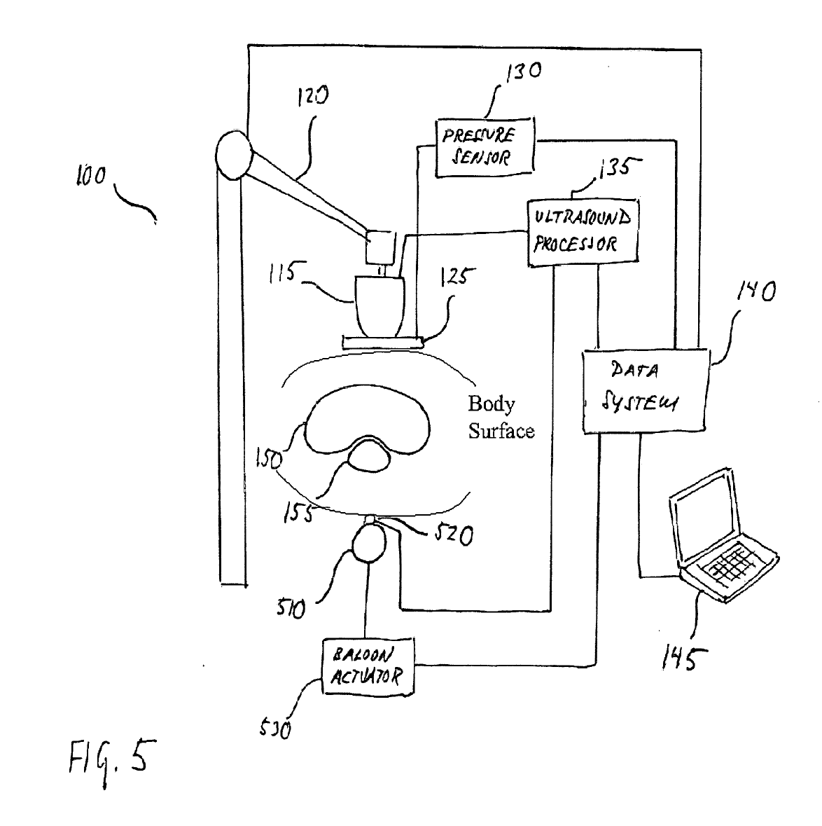

FIG. 5 illustrates another exemplary embodiment of the present invention in which ultrasound strain imaging system 100, which further includes a rectal balloon 510, a balloon actuator 530, and an ultrasound transmitter 530 disposed on the rectal balloon. In this embodiment, the rectal balloon provides a second source of abdominal pressure on the prostate, and the ultrasound transmitter 530 and the transcutaneous ultrasound probe 115 act in conjunction as a transmission-based ultrasound imaging system. The ultrasound transmitter 520 may be connected to the ultrasound processor 135 to operate in conjunction with the transcutaneous ultrasound probe 115. The balloon actuator 530 enables an operator to control the strain exerted on the prostate 155 by controlling the pressure within the rectal balloon 510. The balloon actuator 530 may be connected to the data system 140, and may receive control signals from and transmit data to the software running on the data system 140.

In this embodiment, the ultrasound transmitter 520 and the transcutaneous ultrasound probe 115 act in conjunction as a transmission-based ultrasound imaging system, also referred to as transmission mode. Transmission-based ultrasound is an alternative mode of operation to the pulse/echo mode of operation. In transmission-based ultrasound, one ultrasound probe transmits acoustic energy and another probe receives it. This contrasts with pulse/echo mode, in which one ultrasound probe serves as both transmitter and receiver.

In this embodiment, the ultrasound transmitter 520 transmits acoustic energy that is received by each element of the transcutaneous ultrasound probe 115. By operating in this manner, the flux of the acoustic energy received by each element of the transcutaneous ultrasound probe 115 is much greater than that received in a conventional pulse/echo mode. In pulse/echo mode, which is the conventional operating mode for a typical ultrasound probe, the same probe transmits and receives the same acoustic energy, which is reflected off of acoustic scatterers in the propagation medium. For each scattering event, only a small portion of the reflected energy falls within the field of view of the ultrasound probe elements. Accordingly, only a small portion of the transmitted acoustic energy is received.

By operating in transmission mode, A transmission-based ultrasound system receives directly transmitted acoustic energy, and thus does not suffer from the signal degradation endemic to pulse/echo systems. As such, the transmission-based the system illustrated in FIG. 5 may provide higher quality imagery than that possible in a pulse/echo system; and since signal strength is strong. Further, since the received signal is strong, the software may be able to resolve distances corresponding to the acoustic interfaces corresponding to the prostate 155 to high precision, resulting in precise definition of the contours of the prostate 155.

In order to operate the system 100 as a transmission-based system, as illustrated in FIG. 5, the signals corresponding to the ultrasound transmitter 520 and transcutaneous ultrasound probe 115 must be synchronized. The signals corresponding to the ultrasound transmitter 520 and the transcutaneous ultrasound probe 115 must be synchronized because the time between the transmission of acoustic energy (by the ultrasound transmitter 520) and its reception (by the transcutaneous ultrasound probe 115) must be determined so that the software may use this information to reconstruct signals similar to those illustrated in FIGS. 4A and 4B. Once the software has reconstructed the signals accordingly, it may determine the near and far surfaces of the prostate 155 in a manner similar to that described earlier. It will be apparent to one skilled in the art how to synchronize these signals and reconstruct them in order to operate the system illustrated in FIG. 5 operate the system illustrated in FIG. 5 as a transmission-based system.

Variations to the exemplary embodiment illustrated in FIG. 5 are possible. For instance, the rectal balloon 520 may be the sole source of pressure on the prostate 115, whereby the mechanical arm 120 and the pressure interface 125 may be optional. Further, the ultrasound transmitter 520 may be an ultrasound probe that operates in pulse/echo mode, in which case ultrasound transmitter 520 and the transcutaneous ultrasound probe 115 may operate independently or in conjunction. The ultrasound transmitter 520 may be a pulse/echo mode ultrasound probe, and the transcutaneous utrasound probe 115 may be optional. In this case, the pressure interface 125 provides pressure, and the ultrasound transmitter 520 may be the sole ultrasound probe. Other permutations are possible and within the scope of the invention.

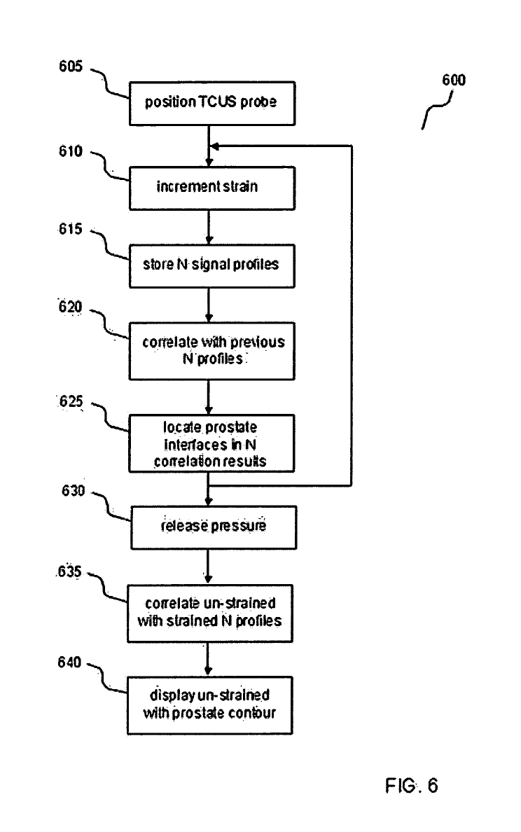

FIG. 6 illustrates an exemplary process 600 for performing ultrasound strain imaging according to the present invention. Process 600 may be implemented by the software, may run in an automated fashion, and may involve operator interaction.

In step 605, the transcutaneous ultrasound probe 115 is positioned over the patient lying on the table of the linear accelerator 105. An operator may position the mechanical arm 130 in such a way that the transcutaneous ultrasound probe 115 and the pressure interface 125 are in contact with, but generatlly not applying pressure to, the patient's abdomen. Once the transcutaneous ultrasound probe 115 is in contact with the patient's abdomen, the ultrasound processor 135 may begin acquiring ultrasound imagery, and the user interface 145 may begin displaying the acquired ultrasound imagery.

The mechanical arm 120 should be positioned such that a vector normal to the surface of the pressure interface 125 (hereinafter “pressure vector”) is should be directed toward the prostate 155 such that as pressure is applied (in a later step), the bladder 150 may conform around the prostate 155 and substantially stabilize it the prostate 155 in a manner described above.

In step 610, the mechanical arm 130 moves the transcutaneous ultrasound probe 115 and the pressure interface 125 along the pressure vector. The motion may be incremental or continuous. If the motion is incremental, an increment size may be selected based on factors such as the pressure of the bladder 150 and the size of the prostate 155. The increment may vary from fractions of millimeters to many millimeters, depending on the patient. by an incremental distance of about 1 mm. As the pressure interface 125 and the transcutaneous ultrasound probe 115 translate along the pressure vector, they exert strain on the bladder and the prostate commensurate with their 1 mm of translation. The mechanical arm 120 may be commanded directly by an operator through the user interface 145, or the software may issue commands to the mechanical arm 120 according to a pre-programmed motion profile.

The mechanical arm 120 may be commanded directly by an operator through the user interface 145, or the software may issue commands to the mechanical arm 120 according to a pre-programmed motion profile.

In step 615, as the pressure interface 125 and the transcutaneous ultrasound probe 115 move along the pressure vector and reach 1 mm of translation, the ultrasound processor 135 acquires image data from the transcutaneous ultrasound probe 115, and transmits the data to the data system 140, wherein the software receives and stores the data values corresponding to the signals acquired by each element in the transcutaneous ultrasound probe 115. As used in to exemplary process 600, the transcutaneous ultrasound probe 115 has N elements, and the data from each may be processed and stored independently. Each of tThe N stored signals may be like those depicted illustrated in FIG. 4A, whereby the most recently stored signal may correspond to signal 415 and a previously stored signal corresponds to signal 410.s. 4A and 4B.

In step 620, the software correlates each of the N signals with the most recently stored corresponding signal. If this is the first incremental motion of the pressure interface 125 and the transcutaneous ultrasound probe 115, then step 620 may be bypassed, since there may be no previous data with which to correlate.

The software correlates the two N signals using processing techniquest that are known to the art, such as circular cross correlation algorithms that are available as functions within commercial mathematical software packages. The result of the correlation may include an N-dimensional array of correlation amplitudes and phases corresponding with each signal data point. The correlation phase corresponds to a time shift between a data point of the current signal and its strongest correlated counterpart in the previous signal; and the correlation amplitude corresponds to the degree of correlation.

If more than one previous signal is stored, the software may repeat the correlation process, but between the current signal and each of the previous signals independently. This may provide for trending information or allow for the identification and removal of outlier correlation results.

In correlating the current and previous signals, the software generally correlates the speckle present in the signal data. Speckle refers to the texture-like features present in ultrasound data, which results from small acoustic scatterers present in the tissue. The observed speckle is in part due to constructive and destructive interference of the acoustic wavefront as it propagates through tissue. Speckle is stable, such that a point in a target tissue that presents speckle in ultrasound imagery will continue to do so for repeated images. Accordingly, speckle results from a spatially distributed low intensity reflecting medium that provides “texture” through which multiple signals may be correlated. In other words, speckle is used to correlate the signals.

If the correlation yields portions of substantially zero correlation between the current and previous signal, it may be due to out-of-plane motion of a speckle scatterer. Since speckle is used to correlate the current and previous signals, if a speckle scatterer undergoes out-of-plane motion, the absence (or sudden presence) of the scatterer from one signal to the next may corrupt the correlation, and result in outliers in the resulting prostate contour. Out-of-plane motion is substantially mitigated by incrementing the pressure interface 125 and transcutaneous ultrasound probe 115 depending on the length of the increment or the speed by which the transcutaneous ultrasound probe 115 and the pressure interface 125 are translated by 1 mm.

In step 625, the acoustic interfaces 330 and 335 are identified by the software. The software searches for sudden changes in correlation phase computed in step 620, and assesses the degree of correlation corresponding to these phase changes by evaluating the correlation amplitude. Since the prostate will respond to strain differently than the surrounding tissue, the difference in response to strain may be apparent in abrupt correlation phase changes at the acoustic interfaces 330 and 335. The software may identify these abrupt changes and store the corresponding distances. The software may identify the abrupt phase changes by comparing the phase differences in phase to a preset threshold, or may be identified by selecting the two highest phase changes within a certain window of time. It will be readily apparent to one of ordinary skill how to implement an algorithm for selecting the phase changes corresponding to acoustic interfaces 330 and 335. Doing this for N signals (one per element) may yield an array of distances corresponding to the contour of the prostate.

Although steps 625 and 630 illustrate the use of speckle correlation to identify changes in elasticity corresponding to the contours of the prostate, other methods are possible and within the scope of the invention. For example, techniques such as vibro-acoustography may be employed to discriminate the contours of the prostate instead of speckle correlation. In vibro-acoustography, the transcutaneous ultrasound probe 115 may transmit continuous acoustic energy in frequencies ranging from 100 Hz to 1 kHz. Each of the N elements in the transcutaneous ultrasound probe 115 receives acoustic energy reflected from the speckle scatters within each of the N fields of view. The software may spectrographically analyze the signals received from each of the N elements to identify Doppler-related shifts in the signals. Doppler-related shifts correspond to changes in elasticity that occur at the near and far surfaces of the prostate 155.

As illustrated in FIG. 6, steps 610-625 are repeated at 1 mm increments over a range of at least 1 cm. It requires approximately 1 cm of translation to induce the bladder 150 to conform around and stabilize the prostate 155 As illustrated in FIG. 6. Any further pressure exerts strain on the prostate, which imposes strain differences between the prostate 155 and the surrounding tissue steps 610-624 may be repeated until the correlation in step 620 yields phase correlations with corresponding correlation amplitudes that are sufficiently high such that the software may decide that the acoustic interfaces 330 and 335 of the prostate have been determined for a sufficient number of the N signals.

The pressure is released in step 630, in which the mechanical arm reverses the translation of the pressure interface 125 and the transcutaneous ultrasound probe 115. This may be done by an operator through the user interface 145, or automatically by the software. On releasing pressure, contact between the transcutaneous ultrasound probe 115 and the abdomen may be such that acoustic coupling between the abdomen and the transcutaneous ultrasound probe 115 is maintained.

In step 635, which is may be optional, the software stores and correlates N signals acquired in which the pressure was released with the N signals stored in the last iteration of step 615, which occurred when the prostate 155 was subjected to the greatest strain. The purpose of this step is to confirm the contours of the prostate 155 in its un-strained state.

In step 640, the user interface displays the un-strained contours of the prostate. Additionally, the software may compute and issue commands to the linear accelerator 105 controller 147 to fine tune its pointing so that the maximum dosage may delivered to the prostate 155 and not to the surrounding tissue.

It may be the case that the prostate 155 may not be visible according to the above-described techniques, because the elasticity of the prostate 155 and its surrounding tissue may be the same. If this is the case, it may be possible to induce a change in elasticity of the prostate so that it may be visible, and its contours defined, by methods and systems according to the present invention. For example, lesions may be deliberately induced in the prostate whereby the lesions may have different strain characteristics that may make them visible under the systems and processes of the present invention. Alternatively, the prostate may be treated so that its elasticity may be made distinct from that of the surrounding tissue. Methods of achieving either of these results include High Intensity Focused Ultrasound (HIFU), which is a noninvasive ultrasound-based method of delivering focused high intensity acoustic energy to impart thermal energy in a localized region within a surrounding tissue, and which is known in the art.

Although the above description refers to prostate cancer treatment, it will be readily apparent to one of ordinary skill that the systems and processes of the present invention may be used for other medical procedures, such as breast cancer treatment or ablative therapy of tumors in a liver. Although the techniques to be applied in these cases may not involve using the bladder as a stabilizing background, there may be geometries in the surrounding anatomy that may assist in stabilizing the tumor being treated, given the direction and force with which the strain is exerted.

It will be apparent to those skilled in the art that various modifications and variation can be made in the present invention without departing from the spirit or scope of the invention. Thus, it is intended that the present invention cover the modifications and variations of this invention provided they come within the scope of the appended claims and their equivalents.

1. A system for providing ultrasound strain imaging comprising:
an ultrasound probe;
a mechanical arm connected to the ultrasound probe;
an ultrasound processor; and
a data system having a computer readable medium encoded with a program for controlling the mechanical arm to apply pressure on a patient's anatomy, and for determining the contours of a target anatomical structure based on a difference between an elasticity of the target anatomical structure and an elasticity of a surrounding tissue.

2. The system of claim 1, further comprising a pressure interface mechanically connected to the ultrasound probe.

3. The system of claim 2, further comprising a pressure sensor mechanically connected to the pressure interface.

4. The system of claim 1, further comprising a linear accelerator having a linear accelerator controller.

5. The system of claim 1, wherein the ultrasound probe is a transcutaneous ultrasound probe.

6. The system of claim 1, further comprising:
a rectal balloon; and
an ultrasound transmitter disposed on the rectal balloon.

7. The system of claim 6, wherein the computer readable medium is encoded with a program for synchronizing a signal corresponding to the ultrasound probe and a signal corresponding to the ultrasound transmitter.

8. The system of claim 1, wherein the program for controlling the mechanical arm to apply pressure on a patient's anatomy, and for determining the contours of a target anatomical structure based on a difference between an elasticity of the target anatomical structure and an elasticity of a surrounding tissue includes:
translating the mechanical arm by plurality of increments;
acquiring ultrasound signals from the ultrasound processor before and after each increment;
correlating the ultrasound signals; and
identifying features in the correlated ultrasound signals corresponding to the target anatomical structure.

9. The system of claim 1, wherein the patient's anatomy is the patient's abdomen.

10. The system of claim 1, wherein the target anatomical structure is a prostate.

11. A method for identifying a contour corresponding to a target anatomical structure in ultrasound imagery, comprising:
positioning an ultrasound probe to be substantially in contact with a patient's anatomy, the ultrasound probe being connected to a mechanical arm;
acquiring a first ultrasound signal from the ultrasound probe;
commanding a mechanical arm to apply pressure to the anatomy;
acquiring a second ultrasound signal from the ultrasound probe;
correlating the first ultrasound signal with the second ultrasound signal;
identifying the contour based on the result of the correlating; and
displaying the contour.

12. The method of claim 11, wherein the target anatomical structure is a prostate.

13. The method of claim 12, wherein the commanding a mechanical arm to apply pressure to the anatomy includes commanding the mechanical arm to translate along a direction from the patient's abdomen toward the prostate.

14. The method of claim 12, wherein commanding the mechanical arm to translate includes commanding the mechanical arm to translate by an increment corresponding to a pressure of the patient's bladder and a size of the prostate.
